(19) United States
(12) Patent Application Publication
(10) Pub. No.: US 2008/0171344 A1
(43) Pub. Date: Jul. 17, 2008

(54) Methods, Kits and Materials for Diagnosing Disease States by Measuring Isoforms or Proforms of Myeloperoxidase

(76) Inventor: Kenneth P. KAPSNER, Minneapolis, MN (US)

Correspondence Address: STERNE KESSLER GOLDSTEIN & FOX, P.L.L.C., 1100 NEW YORK AVENUE, N.W., WASHINGTON, DC, 20005, US

(21) Appl. No.: 11/963,436

(22) Filed: Dec. 21, 2007

Related U.S. Application Data
(60) Provisional application No. 60/876,480, filed on Dec. 22, 2006.

(51) Int. Cl. G01N 33/573; C12Q 1/28

(52) U.S. Cl. 435/7.4; 435/28

(57) ABSTRACT

There exists a need in the art for diagnosing specific disease states through measuring a concentration myeloperoxidase (MPO) isoform and/or proform or a combination thereof in a test sample. The claimed method provides a high specificity assay that has improved diagnostic specificity and improved sensitivity in that it reduces detection of normal MPO present in the sample. Antibodies and kits for performing the described method are further described.

This application claims the benefit of U.S. Provisional Application No. 60/876,480, filed on Dec. 22, 2006, which is herein incorporated by reference in its entirety.

### BACKGROUND

MPO is a tetrameric, heavily glycosylated, basic heme-containing enzyme of approximately 150 kDa found predominantly in polymorphonuclear leukocytes (PMNs) (Miyasaki, K. M. et al., Arch. Biochem. Biophys. 246:751-764 (1986); Wright, J. et al., Biochim. Biophys. Acta 915:68-76 (1987)). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of two identical disulfide-linked protomers, each of which possesses a protoporphyrin-containing 59-64 kDa heavy subunit and a 14 kDa light subunit (Nauseef, W. M, et al., Blood 67:1504-1507 (1986)). Each half molecule contains a covalently bound heme that exhibits unusual spectral properties responsible for the characteristic green color of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. An immature, inactive proform of MPO is also secreted from cells.

Mature MPO is formed from a biosynthetic precursor, apoproMPO, which is processed in the endoplasmic reticulum (ER) (Hansson, M. Arch. Biochem. Biophys. 445:214-224 (2006); Olsson, I. Jpn. J. Infect. Dis. 57:S13-S14 (2004)). Within the ER, apoproMPO undergoes glycosylation and is incorporated with heme to form the enzymatically active proMPO that is exported to the Golgi apparatus where it plays an important role in protein trafficking (Andersson, E., et al., J. Biol. Chem. 273:4747-4753 (1998); Hur, S-J. et al., J. Biol. Chem. 264:8542-8548 (1989)).

MPO is abundant in neutrophils and monocytes, accounting for 5%, and 1 to 2%, respectively, of the dry weight of these cells (Nauseef, W. M, et al., Blood 67:1504-1507 (1986); Hurst, J. K. In: Everse J.; Everse K.; Grisham M. B., eds. Peroxidases in chemistry and biology 1st ed. Boca Raton: CRC Press; 37-62 (1991)). The heme protein is stored in primary azurophilic granules of leukocytes and secreted into both the extracellular milieu and the phagolysosomal compartment following phagocyte activation by a variety of agonists (Klebanoff, S. J, et al. The neutrophil: functions and clinical disorders. Amsterdam: Elsevier Scientific Publishing Co. (1978)). Immunohistochemical methods have demonstrated that MPO is present in human atherosclerotic lesions. It is believed that MPO isoforms or proforms have not yet been reported to be present at increased levels in blood samples from individuals with atherosclerosis. However, MPO isoforms have been shown to be secreted by neutrophils (Miyasaki, K. T., et al, Anal. Biochem. 193:38-44 (1991)).

PMNs are of particular importance for combating infections. These cells contain MPO, with well documented microbicidal action. PMNs act nonspecifically by phagocytosis to engulf microorganisms, incorporate them into vacuoles, termed phagosomes, which fuse with granules containing myeloperoxidase to form phagolysosomes. In phagolysosomes, the enzymatic activity of the myeloperoxidase leads to the formation of hypochlorous acid, a potent bactericidal compound. Macrophages are large phagocytic cells which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease, Parkinson's disease and stroke as well as other inflammatory diseases or conditions, such as chronic obstructive pulmonary disease, cystic fibrosis, atherosclerosis, inflammatory bowel disease, renal glomerular damage and rheumatoid arthritis. Lung cancer has also been suggested to be associated with high MPO levels. In addition, levels of native MPO were previously shown to correlate with cardiovascular disease (U.S. Pat. Pub. No. 2002/0164662). However, none of these previous studies have shown specific correlation of the presence of MPO isoforms and/or proforms with specific disease states.

### SUMMARY

A need in the art exists for a diagnostic assay that can specifically detect the presence of MPO isoforms and/or proforms so that a positive identification can be made as to the presence of an allergic, inflammatory, cardiovascular or autoimmune disease state.

The claimed method provides a high specificity assay that has improved diagnostic specificity and improved sensitivity in that it differentially detects at least one myeloperoxidase (MPO) isoform or proform or combination thereof in a test sample, wherein the method includes:

(a) contacting said at least one MPO isoform or proform or combination thereof with at least one capture molecule that specifically binds with said at least one MPO isoform or proform or combination thereof; and

(b) detecting capture of said at least one MPO isoform or proform or combination thereof with at least one detector molecule; in order to aid in diagnosis or prognosis of an allergic, inflammatory, cardiovascular or autoimmune disease condition. Antibodies and kits for performing the described method are further described.

Further embodiments, features, and advantages of the disclosure, as well as the structure and operation of the various embodiments, are described in detail below with reference to the accompanying figures.

### BRIEF DESCRIPTION OF THE FIGURES

The accompanying figures, which are incorporated herein and form a part of the specification, illustrate one or more embodiments of the disclosure and, together with the description, further serve to explain the principles of the disclosure.

FIG. 1A-B shows that certain antibody pairs (MPO-A; FIG. 1A and MPO-C; FIG. 1B) differ significantly in the measurement of different patient populations, especially ambulatory normal patients. Normal females (open box), normal males (open diamond), troponin positive (open triangle), rheumatoid arthritis (X), multiple sclerosis (+), Alzheimer's (open circle).

FIG. 2 shows the ion-exchange column separation of MPO isoforms or proforms from Troponin I positive and ambulatory normal samples using the MPO-A assay format. Troponin I patient 1 (open box), Troponin I patient 2 (open diamond), ambulatory patient 1 (open triangle), ambulatory patient 2 (*).

FIG. 3 shows the ion-exchange column separation of neutrophil (NMPO) and ambulatory normal MPO isoforms or proforms using the MPO-A or MPO-C assay format. MPO-A NMPO (open box), MPO-C NMPO (open diamond), MPO-A normal 1 sample (open triangle).

FIG. 4 shows the ion-exchange column separation of MPO isoforms or proforms from ambulatory normal pooled samples using the MPO-A or MPO-C assay format or a commercially available kit (ALPCO). MPO-A Npool (open box), MPO-C NPool (open diamond), ALPCO NPool (open triangle).

### DETAILED DESCRIPTION

### Overview

A new diagnostic/prognostic assay for the presence of MPO isoforms and/or proforms is provided below.

In one embodiment, the disclosure relates to a diagnostic test for assessing whether a patient has an allergic, inflammatory, cardiovascular or autoimmune disease. In another embodiment, the disclosure relates to a prognostic test for assessing a patient's risk for developing an allergic, inflammatory, cardiovascular or autoimmune disease. In certain aspects, the methods comprise differentially detecting MPO isoforms and/or proforms in a test sample obtained from the individual.

It should be noted that as used herein and in the appended claims, the singular forms “a,” “and,” and “the” include plural referents unless the context clearly dictates otherwise.

Although any methods, devices and materials similar or equivalent to those described herein can be used in the practice of the invention as presented in the claims, the particular methods, devices and materials are now described.

In one embodiment, the disclosure provides a method to aid in diagnosis or prognosis of an allergic, inflammatory, cardiovascular or autoimmune disease condition by differentially detecting at least one, but not all myeloperoxidase (MPO) isoforms or proforms or combination thereof in a test sample, the method comprising:
(a) contacting said at least one MPO isoform or proform or combination thereof with at least one capture molecule that specifically binds with said at least one, but not all MPO isoforms or proforms or combination thereof, and
(b) detecting capture of said at least one, but not all MPO isoforms or proforms or combination thereof with a detector molecule.

In further embodiments, the method provides differential detection of at least one myeloperoxidase (MPO) isoform or proform or combination thereof without detection of at least one other myeloperoxidase (MPO) isoform or proform or combination. In certain embodiments, the method provides for differential detection of one or more MPO isoforms without detection of at least one MPO proform.

In another embodiment, the captured MPO isoform or proform or combination thereof is detected either by a detector molecule that specifically binds with said at least one MPO isoform or proform or combination thereof, or by competition with a labeled MPO isoform or proform or combination thereof. Generally, the detector molecule does not bind at least one other MPO isoform or proform. In at least one embodiment, the detector molecule specifically binds with said at least one MPO isoform, but does not bind at least one MPO proform. In various embodiments, MPO isoforms specifically bound by the detector molecule are active forms. In various further embodiments, at least one MPO proform not bound by the detector molecule is an inactive form.

In a further embodiment, the specific detection of at least one MPO isoform or proform or combination thereof is correlated to an allergic, inflammatory, cardiovascular or autoimmune disease. In one embodiment, the inflammatory disease is inflammatory bowel disease. In another embodiment, the autoimmune disease is selected from the group consisting of acute disseminated encephalomyelitis, Addison's disease, ankylosing spondylitisis, antiphospholipid antibody syndrome, aplastic anemia, autoimmune hepatitis, coeliac disease, Crohn's disease, diabetes mellitus, Goodpasture's syndrome, Graves' disease, Hashimoto's disease, idiopathic thrombocytopenic purpura, lupus erythematosus, multiple sclerosis, myasthenia gravis, optic neuritis, pemphigus, primary biliary cirrhosis, rheumatoid arthritis, Reiter's syndrome, Sjogren's syndrome, warm autoimmune hemolytic anemia and Wegener's granulomatosis.

In another embodiment, the capture molecule of the disclosure is selected from a monoclonal antibody, a mono-specific polyclonal antibody, a binding fragment of an antibody or an antibody mimic. In a further embodiment, the capture molecule is coupled to a solid support comprising a protein coupling surface. The protein coupling surface may be a microtiter plate, a colloidal metal particle, an iron oxide particle, or a polymeric bead.

In one embodiment, the detector molecule comprises a monoclonal antibody, a mono-specific polyclonal antibody, a binding fragment of an antibody, or an antibody mimic. In a further embodiment, the detector molecule comprises a detectable label. The detectable label can include a chemiluminescent agent, a colorimetric agent, an energy transfer agent, an enzyme, a substrate of an enzyme reaction, a fluorescent agent or a radioisotope. In yet another embodiment, the enzyme may be alkaline phosphatase, amylase, luciferase, catalase, beta-galactosidase, glucose oxidase, glucose-6-phosphate dehydrogenase, hexokinase, horseradish peroxidase, lactamase, urease, or malate dehydrogenase. In one embodiment, the detector molecule is coupled to a detectable label or to a secondary detector molecule that is coupled to a detectable label.

In one embodiment, the method for diagnosis/prognosis is an immunoassay. In a further embodiment, the immunoassay is a solid-phase immunoassay.

In one embodiment, the test sample may be whole blood, blood plasma, serum, saliva, cerebral spinal fluid, urine, amniotic fluid, interstitial fluid, feces, mucus, cell extracts, or tissue extracts.

The disclosure also provides an isolated monoclonal antibody, mono-specific polyclonal antibody, binding fragment of an antibody, or antibody mimic that specifically binds with a proform and/or isoform of MPO.

The disclosure also provides for a kit to aid in diagnosis or prognosis of an allergic, inflammatory, cardiovascular or autoimmune disease condition by measuring at least one MPO isoform or proform or combination thereof in a test sample comprising: at least one capture molecule that specifically binds with at least one MPO isoform or proform or combination thereof and at least one detector molecule. In certain embodiments, the detector molecule is either (a) a molecule that binds with at least one MPO isoform or proform or combination thereof and that is coupled directly or indirectly with a detectable label so as to detect capture of said at least one MPO isoform or proform or combination thereof or (b) a labeled MPO isoform or proform or combination thereof.

In one embodiment, the kit aids in the diagnosis or prognosis of a disease condition including allergic, inflammatory, cardiovascular or autoimmune diseases. In one embodiment, the inflammatory disease is inflammatory bowel disease. In another embodiment, the autoimmune disease is acute disseminated encephalomyelitis, Addison's disease, ankylosing spondylitisis, antiphospholipid antibody syndrome, aplastic anemia, autoimmune hepatitis, coeliac disease, Crohn's disease, diabetes mellitus, Goodpasture's syndrome, Graves' disease, Hashimoto's disease, idiopathic thrombocytopenic purpura, lupus erythematosus, multiple sclerosis, myasthenia gravis, optic neuritis, pemphigus, primary biliary cirrhosis, rheumatoid arthritis, Reiter's syndrome, Sjogren's syndrome, warrn autoimmune hemolytic anemia and Wegener's granulomatosis.

In another embodiment, the kit also contains a calibrator formulation for quantitation of at least one MPO isoform or proform or combination thereof in a test sample. In one embodiment, calibration formulations may be one or more pre-prepared calibrator. In another embodiment, the calibration formulation may be a material capable of being reconstituted to form one or more calibrators in a buffer.

In one embodiment, the kit contains a capture molecule. Capture molecules suitable for use in embodiments of the disclosure include, but are not limited to, a monoclonal antibody, a mono-specific polyclonal antibody, a binding fragment of an antibody, or an antibody mimic. In a further embodiment, the capture molecule is coupled to a solid support. Solid supports suitable for use in embodiments of the disclosure have a protein coupling surface. Such solid supports include, but are not limited to, a microtiter plate, a colloidal metal particle, an iron oxide particle, and a polymeric bead.

In one embodiment, the detector molecule comprises a monoclonal antibody, a mono-specific polyclonal antibody, a binding fragment of an antibody, or an antibody mimic. In another embodiment, the detector molecule is at least one labeled MPO isoform or proform or combination thereof. In a further embodiment, the detector molecule further comprises a detectable label. The detectable label can include a chemiluminescent agent, a colorimetric agent, an energy transfer agent, an enzyme, a substrate of an enzyme reaction, a fluorescent agent or a radioisotope. In yet another embodiment, the enzyme may be alkaline phosphatase, amylase, luciferase, catalase, beta-galactosidase, glucose oxidase, glucose-6-phosphate dehydrogenase, hexokinase, horseradish peroxidase, lactamase, urease or malate dehydrogenase. In one embodiment, the detector molecule is coupled to a detectable label or to a secondary detector molecule that is coupled to a detectable label.

### Myeloperoxidase

In certain embodiments, the disclosure relates to the determination of MPO isoform and/or proform levels in a test sample of a mammal and using said levels as a factor for diagnosing or prognosing an allergic, inflammatory, cardiovascular or autoimmune disease condition in the mammal. In preferred embodiments, mature and inactive MPO are not detected. In these approaches, detection of a MPO isoform and/or proform level that is altered or elevated compared to comparable samples from normal individuals is a factor favoring diagnosis or prognosis of a disease condition.

The term “elevated levels” or “higher levels” as used herein refers to levels of an analyte, such as an MPO isoform and/or proform, that are higher than what would normally be observed in a comparable test sample from control or normal subjects. In some embodiments of the disclosure “control levels” (i.e. normal levels) refers to a range of MPO isoform and/or proform levels that would normally be expected to be observed in a test sample that does not have a disease condition and “elevated levels” refers to MPO isoform and/or proform levels that are above the range of control levels.

The differential levels of various MPO isoforms and/or proforms, or combinations thereof are generally determined using capture molecules. As used herein, the term “capture molecule” refers to any molecule or complex of molecules capable of binding with a MPO isoform or proform of the disclosure under suitable conditions. The selection of those conditions is well known, as well as techniques to vary or modify the binding conditions. For example, it is well known that temperature, pH and time of incubation all play a role in binding. Generally, the binding occurs with sufficient specificity to exclude significant binding to more than one ligand. But it is understood that “capture molecules” that specifically bind more than one MPO isoform or proform may be used in such assays. In some embodiments of the disclosure, the “capture molecule” is an antibody or ligand binding fragment or analog thereof. The “capture molecule” may also be other proteins or nucleic acids, or portions or analogs thereof, that bind MPO isoforms and/or proforms in the practice of certain embodiments of the disclosure.

Preferential or specific binding of a capture molecule is characterized by binding to one MPO isoform but not binding to a second (different) MPO isoform or proform under the same or similar binding conditions. In some embodiments, a capture molecule may have measurably higher binding affinity for one MPO isoform or possibly one, two or three MPO isoform, but have significantly lower binding affinity for other antigens, including other isoforms and/or proforms of MPO. For example, an antibody may bind to one MPO isoform or possibly one, two or three MPO isoform, with at least 5 fold, at least 10 fold, at least 20 fold, or at least 100 fold greater affinity than other non-target forms of MPO. One would recognize that capture molecules may preferentially or specifically bind more than one MPO isoform or proform or combination thereof, based on the presence of cross-reactive binding regions located on the MPO isoform or proform or combination thereof.

Using certain embodiments of the disclosure, one may associate altered levels of at least one MPO isoforms or proforms or combinations thereof with allergic, inflammatory, cardiovascular and autoimmune disease conditions. The altered levels of MPO isoforms or proforms or combinations thereof can be associated with the presence of a disease condition in a test sample, or can be used as a prognostic marker for the development of said disease condition in the future. Examples of autoimmune disease conditions include, but are not limited to, acute disseminated encephalomyelitis, Addison's disease, ankylosing spondylitisis, antiphospholipid antibody syndrome, aplastic anemia, autoimmune hepatitis, coeliac disease, Crohn's disease, diabetes mellitus, Goodpasture's syndrome, Graves' disease, Hashimoto's disease, idiopathic thrombocytopenic purpura, lupus erythematosus, multiple sclerosis, myasthenia gravis, optic neuritis, pemphigus, primary biliary cirrhosis, rheumatoid arthritis, Reiter's syndrome, Sjogren's syndrome, warm autoimmune hemolytic anemia and Wegener's granulomatosis.

### Preparation of the Test Sample

The terms “test sample”, “patient sample” or “body sample” are used interchangeably herein. The test sample in the diagnostic method can include, for example, whole blood, blood plasma, urine, serum, saliva, cerebral spinal fluid, urine, amniotic fluid, interstitial fluid, feces, mucus, body tissue extracts or cellular extracts. The whole blood can be obtained from the individual or test subject using standard clinical procedures. Plasma can be obtained from whole blood samples by centrifugation of anti-coagulated blood. Such process provides a bottom layer of packed red blood cells, an intermediate buffy coat layer and a supernatant plasma layer.

Serum can be collected by centrifugation of whole blood samples that have been collected in tubes that are free of anti-coagulant. The blood is permitted to clot prior to centrifugation. The yellowish or yellowish-reddish fluid that is obtained by centrifugation is the serum.

Leukocytes can be isolated from whole blood samples by any of various techniques including buoyant density centrifugation and others known in the art.

The test sample may also be a clinical cytology specimen (e.g., fine needle breast biopsy or pulmonary cytology specimen) or a tissue specimen from, for example, stomach, lung, breast, ovarian, pancreatic, or heart. The tissue specimen may be fresh or frozen.

Test samples may be obtained from a human person or a commercially significant mammal, including but not limited to a cow or horse. Test samples may also be obtained from household pets, including but not limited to a dog or cat.

### Antibodies of the Disclosure

Isoforms or proforms of MPO can be used to raise polyclonal and monoclonal antibodies. The antibodies of the disclosure therefore preferentially bind MPO isoforms and/or proforms and do not bind mature MPO. In addition, as discussed in more detail below, the antibodies may be pan-specific antibodies. The term “pan-specific antibody” as used herein refers to an antibody that selectively binds at least two members of a group of related polypeptides (e.g. MPO isoforms or proforms) and does not selectively bind polypeptides outside the group of related polypeptides.

The term “antibody” as used herein refers to immunoglobulin molecules and immunologically active portions of immunoglobulin (Ig) molecules. Such antibodies include, but are not limited to, polyclonal, monoclonal, mono-specific polyclonal antibodies, antibody mimics, chimeric, single chain, Fab, Fab′ and F(ab′)₂ fragments, Fv, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain. Reference herein to antibodies includes a reference to all such classes, subclasses and types of antibody species.

Preferably, an antibody molecule shows little or no binding to non-MPO isoform or proform-expressing cells. In some embodiments, the antibody also shows no binding to mature MPO. The reactivity of an antibody molecule with a target antigen may be determined in methods of the disclosure by any appropriate means. Suitable protocols are well known in the literature (see for example Antibodies: A Laboratory Manual E. Harlow and D. Lane, Cold Spring Harbor Laboratory Press, NY, 1988). Tagging with individual detector molecules is one possibility. The detector molecules may directly or indirectly generate detectable, and preferably measurable, signals. The linkage of detector molecules may be directly or indirectly, covalently, e.g., via a peptide bond or non-covalently. Linkage via a peptide bond may be as a result of recombinant expression of a gene fusion encoding antibody and detector molecule. The actual mode of determining the binding of an antibody molecule is not a feature of the disclosure and those skilled in the art are able to choose a suitable mode according to their preference and general knowledge.

It has been shown that fragments of an antibody can perform the function of binding antigens. As used herein “antigen binding fragments” includes, but is not limited to: (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′)₂ fragments (vii) single chain Fv molecules (scFv), wherein a VH domain and a VL domain are linked by a peptide linker which allows the two domains to associate to form an antigen binding site (Bird, et al., Science 242:423-426 (1988); Huston et al., Proc. Natl. Acad. Sci. USA 85:5879-5883 (1988)); (viii) bispecific single chain Fv dimers (PCT/US92/09965) and (ix) diabodies, multivalent or multispecific fragments constructed by gene fusion (WO94/13804; P. Holliger et al., Proc. Natl. Acad. Sci. USA 90:6444-6448 (1993)).

A MPO isoform or proform may be intended to serve as an antigen, or a portion or fragment thereof, and additionally can be used to generate antibodies that immunospecifically bind to an epitope within the antigen, using standard techniques for polyclonal and monoclonal antibody preparation. In some embodiments, the antigenic peptide comprises at least 10 amino acid residues, or at least 15 amino acid residues, or at least 20 amino acid residues, or at least 30 amino acid residues.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

That an antibody “selectively binds” or “specifically binds” means that the antibody reacts or associates more frequently, more rapidly, with greater duration, with greater affinity, or with some combination of the above to an epitope than with alternative substances, including unrelated proteins. “Selectively binds” or “specifically binds” means, for instance, that an antibody binds to a protein with a KD of at least about 0.1 mM, but more usually at least about 1 μM. “Selectively binds” or “specifically binds” means at times that an antibody binds to a protein at times with a KD of at least about 0.1 μM or better, and at other times at least about 0.01 μM or better.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

MPO isoform and/or proform specific antibodies can be acquired by a number of methods known in the art, including panning and affinity chromatography. For the production of polyclonal antibodies, various suitable host animals (e.g., rabbit, goat, mouse or other mammal) may be immunized by one or more injections with MPO isoforms or proforms, synthetic variants, derivatives, or fragments thereof. An appropriate immunogenic preparation can contain, for example, the naturally occurring immunogenic isoforms or proforms, a chemically synthesized polypeptide representing the immunogenic isoforms or proforms, or a recombinantly expressed polypeptide of the immunogenic isoforms or proforms, or fragments thereof. Furthermore, the protein may be conjugated to a second protein known to be immunogenic in the mammal being immunized. Examples of such immunogenic proteins include but are not limited to keyhole limpet hemocyanin, serum albumin, bovine thyroglobulin and soybean trypsin inhibitor. The preparation can further include an adjuvant. Various adjuvants used to increase the immunological response include, but are not limited to, Freund's (complete and incomplete), mineral gels (e.g., aluminum hydroxide), MPL-TDM (monophosphoryl Lipid A-synthetic trehalose dicorynomycolate), and PROVAX™ (Biogen Idec).

Polyclonal antibodies directed against MPO isoforms or proforms can be prepared by any known method. Polyclonal antibodies are raised by immunizing an animal (e.g. a rabbit, rat, mouse, donkey, etc) by multiple subcutaneous or intraperitoneal injections of the relevant MPO isoform or proform (a purified peptide fragment, full-length recombinant protein, fusion protein, etc) optionally conjugated to keyhole limpet hemocyanin (KLH), serum albumin, etc. diluted in sterile saline and combined with an adjuvant (e.g. Complete or Incomplete Freund's Adjuvant) to form a stable emulsion. The polyclonal antibody is then recovered from blood, ascites and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

While the resulting antibodies may be harvested from the serum of the mammal to provide polyclonal preparations, it is often desirable to isolate individual lymphocytes from the spleen, lymph nodes or peripheral blood, to provide homogenous preparations of monoclonal antibodies. In some embodiments, the lymphocytes are obtained from the spleen.

“Monoclonal antibodies” (“mAbs”) as used herein, refers to a population of antibody molecules that contain only one molecular species of antibody molecule consisting of a unique light chain gene product and a unique heavy chain gene product. In particular, the complementarity determining regions of the mAb are identical in all the molecules of the population. mAbs, thus contain an antigen binding site capable of immunoreacting with a particular epitope of the antigen characterized by a unique binding affinity for it.

In this well known process (Kohler et al., Nature 256:495 (1975)), the relatively short-lived, or mortal, lymphocytes from a mammal which has been injected with antigen are fused with an immortal tumor cell line (e.g. a myeloma cell line), thus producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody.

Hybridoma cells thus prepared are seeded and grown in a suitable culture medium that contains one or more substances that inhibit the growth or survival of the unfused, parental myeloma cells. Those in the art will appreciate that reagents, cell lines and media for the formation, selection and growth of hybridomas are commercially available from a number of sources and standardized protocols are well established. Generally, culture medium in which the hybridoma cells are growing is assayed for production of mAbs against the desired antigen. In some embodiments, the binding specificity of the monoclonal antibodies produced by hybridoma cells is determined by immunoprecipitation or by an in vitro assay, such as a radioimmunoassay (RIA) or enzyme-linked immunosorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein A, hydroxylapatite chromatography, gel electrophoresis, dialysis or affinity chromatography.

As used herein the term “modified antibody” shall be held to mean any antibody, or antigen binding fragment or recombinant thereof, immunoreactive with an MPO isoform and/or proform or combination thereof in which at least a fraction of one or more of the constant region domains has been deleted or otherwise altered so as to provide desired biochemical characteristics such as altered serum half-life when compared with a whole, unaltered antibody of approximately the same binding specificity. In some embodiments, the modified antibodies of the disclosure have at least a portion of one of the constant domains deleted.

Various techniques have been developed for the production of antibody fragments. Traditionally, fragments are derived via proteolytic digestion of intact antibodies (see, e.g., Morimoto et al., J. Biochem, Biophys, Methods 24:107-117 (1992); and Brennan et al., Science, 229:81 (1985)). However, fragments can now be produced directly by recombinant host cells. Fab, Fv and ScFv antibody fragments can all be expressed in and secreted from E. coli, allowing the facile production of large amounts of these fragments. Alternatively, Fab′-SH fragments can be directly recovered from E. coli and chemically coupled to form F(ab′)₂ fragments (Carter et al., Bio/Technology 10:163-167 (1992)). In another approach, F(ab′)₂ fragments can be isolated directly from recombinant host cell culture. Fab and F(ab′)₂ fragments with increased in vivo half-life comprising a salvage receptor binding epitope residues are described in U.S. Pat. No. 5,869,046. Other techniques for the production of antibody fragments will be apparent to the skilled practitioner. An antibody of choice can be a single chain Fv fragment (scFv). See WO 93/16185; U.S. Pat. No. 5,571,894; and U.S. Pat. No. 5,587,458. Fv and sFv are the only species of fragments with intact combining sites that are devoid of constant regions; thus, are suitable for reduced nonspecific binding during in vivo use. sFv fusion proteins may be constructed to yield fusion of an effector protein at either the amino or the carboxy terminus of an sFv. The antibody fragment may also be a “linear antibody”, e.g., as described in U.S. Pat. No. 5,641,870. Such linear antibody fragments may be monospecific or bispecific.

It is understood that the compositions and methods of the disclosure are useful for diagnosing a multitude of allergic, inflammatory, cardiovascular or autoimmune disease conditions. As discussed above, the modified antibodies of the disclosure are immunoreactive with MPO isoforms and/or proforms. More generally, modified antibodies useful in certain embodiments of the disclosure may be obtained or derived from any antibody (including those previously reported in the literature) that reacts with MPO isoforms and/or proforms. Further, the parent or precursor antibody, or fragment thereof, used to generate the disclosed modified antibodies may be murine, human, chimeric, humanized, non-human primate or primatized. In other embodiments, the modified antibodies of the disclosure may comprise single chain antibody constructs (such as that disclosed in U.S. Pat. No. 5,892,019, which is incorporated herein by reference) having altered constant domains as described herein. Consequently, any of these types of antibodies modified in accordance with the teachings herein are compatible with this disclosure.

In other compatible embodiments, DNA encoding the desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a source of such DNA. Once isolated, the DNA may be placed into expression vectors, which are then transfected into prokaryotic or eukaryotic host cells such as E. Coli cells, simian COS cells, Chinese Hamster Ovary (CHO) cells or myeloma cells that do not otherwise produce immunoglobulins. More particularly, the isolated DNA (which may be modified as described herein) may be used to clone constant and variable region sequences for the manufacture of antibodies as described in Example 2 of Newman et al., U.S. Pat. No. 5,658,570, which is incorporated by reference herein. Essentially, this entails extraction of RNA from the selected cells, conversion to cDNA, and amplification thereof by PCR using immunoglobulin specific primers. As will be discussed in more detail below, transformed cells expressing the desired antibody may be grown up in relatively large quantities to provide clinical and commercial supplies of the immunoglobulin.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

It will also be appreciated that DNA encoding antibodies or antibody fragments may also be derived from antibody phage libraries as set forth, for example, in the Examples of U.S. Pat. No. 5,969,108 which are incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. Such procedures provide viable alternatives to traditional hybridoma techniques for the isolation and subsequent cloning of monoclonal antibodies and, as such, are clearly within the purview of this disclosure.

Yet other embodiments of the disclosure comprise the generation of substantially human antibodies in transgenic animals (e.g., mice) that are incapable of endogenous immunoglobulin production (see e.g., the Examples of U.S. Pat. Nos. 6,075,181, 5,939,598, 5,591,669 and 5,589,369 which are incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array in such germ line mutant mice will result in the production of human antibodies upon antigen challenge. Another means of generating human antibodies using SCID mice is disclosed in the Examples of U.S. Pat. No. 5,811,524, which are incorporated herein by reference. It will be appreciated that the genetic material associated with these human antibodies may also be isolated and manipulated as described herein.

It will further be appreciated that the scope of this disclosure encompasses all alleles, variants and mutations of the DNA sequences described herein.

According to certain embodiments of the disclosure, techniques can be adapted for the production of single-chain antibodies specific to a polypeptide of the disclosure (see U.S. Pat. No. 4,946,778, the methods of which are incorporated herein by reference). In addition, methods can be adapted for the construction of Fab expression libraries (Huse, et al., Science 246:1275-1281 (1989)) to allow rapid and effective identification of monoclonal Fab fragments with the desired specificity for MPO isoforms or proforms, or derivatives, fragments, analogs or homologs thereof.

For the purposes of the disclosure, it should be appreciated that modified antibodies may comprise any type of variable region that provides for the association of the antibody with MPO isoforms and/or proforms. In this regard, the variable region may comprise or be derived from any type of mammal that can be induced to mount a humoral response and generate immunoglobulins against the desired MPO isoform or isoforms, with or without binding affinity for proforms. As such, the variable region of the modified antibodies may be, for example, of human, murine or non-human primate (e.g. cynomolgus monkeys, macaques, etc.) origin. In some embodiments both the variable and constant regions of the modified immunoglobulins are human. In other selected embodiments, the variable regions of compatible antibodies (usually derived from a non-human source) may be engineered or specifically tailored to improve the binding properties or reduce the immunogenicity of the molecule. In this respect, variable regions useful in certain embodiments of the disclosure may be humanized or otherwise altered through the inclusion of imported amino acid sequences.

Human antibodies can also be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

The present disclosure also encompasses bispecific antibodies that specifically recognize a MPO isoform or proform. Bispecific antibodies are antibodies that are capable of specifically recognizing and binding at least two different epitopes. The different epitopes can either be within the same molecule (e.g. the same MPO isoform or proform) or on different molecules such that both, for example, the antibodies can specifically recognize and bind multiple MPO isoforms or proforms. Bispecific antibodies can be intact antibodies or antibody fragments. Antibodies with more than two valencies are also contemplated. For example, trispecific antibodies can be prepared (Tutt et al., J. Immunol. 147:60 (1991)).

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, K. S, and Chiswell, D. J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also, U.S. Pat. Nos. 5,565,332 and 5,573,905.

According to the present disclosure, techniques can be adapted for the production of single-chain antibodies specific to a MPO isoform or proform (see U.S. Pat. No. 4,946,778). In addition, methods can be adapted for the construction of Fab expression libraries (Huse, et al., Science 246:1275-1281 (1989)) to allow rapid and effective identification of monoclonal Fab fragments with the desired specificity for a MPO isoform or proform, or derivatives, fragments, analogs or homologs thereof. Antibody fragments that contain the idiotypes to a MPO isoform or proform may be produced by techniques in the art including, but not limited to: (a) an F(ab′)2 fragment produced by pepsin digestion of an antibody molecule; (b) an Fab fragment generated by reducing the disulfide bridges of an F(ab′)2 fragment, (c) an Fab fragment generated by the treatment of the antibody molecule with papain and a reducing agent, and (d) Fv fragments.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present disclosure. Heteroconjugate antibodies are composed of two covalently joined antibodies. It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

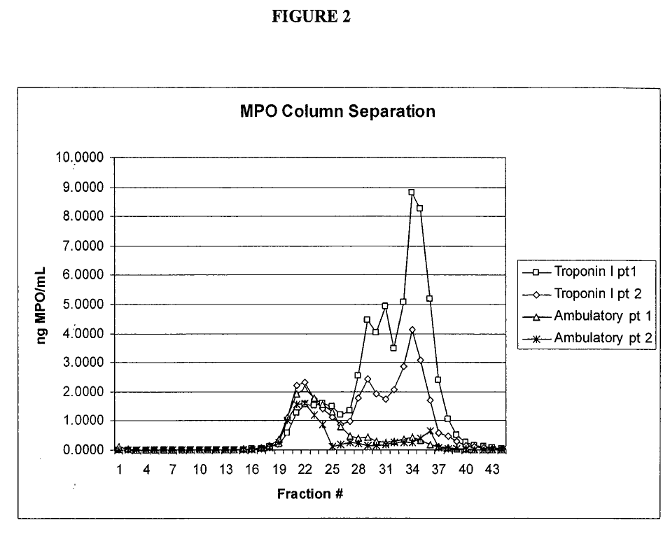

Using the above-described techniques, antibodies can be generated with a desired specificity. For example, a pan-specific antibody to all MPO isoforms and proforms can be generated. In addition, an antibody specific for one or more MPO isoforms and/or proforms can be identified. For example, an antibody can be generated that is specific for MPO isoforms 1 and 3, but has little or no reactivity to isoform 2 (see e.g., FIG. 2). An antibody can be generated that is specific for an MPO proform, but has little or no reactivity to any isoform. An antibody can also be identified that is specific for only one MPO isoform or proform.

Antibodies with the desired specificity can be identified using methods known in the art such as ELISA, ELISpot, western blotting and immunoprecipitation.

### Immunoassay

Assay systems utilizing a capture molecule and a detector molecule to quantify captured molecules are well known. Examples of immunoassays useful in the disclosure include, but are not limited to, radioimmunoassay (RIA), fluoroluminescence assay (FLA), chemiluminescence assay (CA), enzyme-linked immunosorbant assay (ELISA) and the like. See, for example, Johnstone and Thorpe, 1996, In: Blackwell, Immunochemistry in Practice, 3rd ed. (Blackwell Publishing, Malden, Mass.); Ausbul et al., eds., 2003, Current Protocols in Molecular Biology, Wiley & Sons (Hoboken, N.J.); Ghindilis et al., eds., 2003, Immunoassay Methods and Protocols, (Blackwell Publishing, Malden, Mass.); and U.S. Patent Publication No. 20030044865. The immunoassay can be a solid phase assay, a liquid phase assay, and the like.

The immunoassay, in one embodiment, may be designed for an automated, high-throughput instrument. For example, the Access® family of instruments by Beckman Coulter, Inc. are well suited to effectuate the immunoassay of the disclosure. The Access® Immunoassay System allows for rapid throughput of up to 100 tests per hour through the use of a reaction vessel loader that has the capacity for up to 3 hours of continuous sample processing.

In another embodiment, the quantity of target molecule bound to the capture molecule can be determined using a competitive binding assay. In one embodiment, a capture molecule and a horseradish peroxidase (HRP)-labeled MPO isoform and/or proform or combination thereof are used to determine analyte concentration. At least one MPO isoform and/or proform present in the test sample competes for capture molecule binding sites. After removal of unbound label by washing, the HRP signal can be expressed. HRP may be detected by reacting HRP with a calorimetric substrate and measuring the optical density of the reacted substrate at a 450 nm absorbance. The concentration of MPO isoform and/or proform or combination thereof present in the test sample will be inversely proportional to the signal expressed.

For a solid-phase immunoassay, the capture molecule (e.g. a mAb) is immobilized to a solid support. Immobilization conventionally is accomplished by insolubilizing the capture molecule either before the assay procedure, as by adsorption to a water-insoluble matrix or surface (U.S. Pat. No. 3,720,760) or as by non-covalent or covalent coupling (for example, using glutaraldehyde or carbodiimide cross-linking, with or without prior activation of the support with, e.g., nitric acid and a reducing agent as described in U.S. Pat. No. 3,645,852 or in Rotmans et al.; J. Immunol. Methods, 57:87-98 (1983)), or afterward, e.g., by immunoprecipitation.

The solid phase used for immobilization may be any inert support or carrier that is essentially water insoluble and useful in immunometric assays, including supports in the form of, e.g., surfaces, particles, porous matrices, etc. Examples of commonly used supports include small sheets, SEPHADEX® gels, polyvinyl chloride, plastic beads, and assay plates or test tubes manufactured from polyethylene, polypropylene, polystyrene, and the like, including 96-well microtiter plates, as well as particulate materials such as filter paper, agarose, cross-linked dextran, and other polysaccharides. Capture molecules can also be immobilized on a substrate, such as a polymeric bead, colloidal metal or iron oxide particle. Beads can be plastic, glass, or any other suitable material, typically in the 1-20 micron range. In some embodiments, paramagnetic beads are used. Colloidal metal particles such as colloidal gold and silver particles and iron oxide particles can be prepared using many different procedures commercially available or otherwise known to those skilled in the art.

Alternatively, reactive water-insoluble matrices such as cyanogen-bromide-activated carbohydrates and the reactive substrates described in U.S. Pat. Nos. 3,969,287; 3,691,016; 4,195,128; 4,247,642; 4,229,537; and 4,330,440 may be used for capture molecule immobilization. In one embodiment, the immobilized capture molecules are coated on a microtiter plate, and in another embodiment the solid phase is a multi-well microtiter plate that can analyze several samples at one time.

The solid phase is coated with the capture molecules as defined above, which may be linked by a non-covalent or covalent interaction or physical linkage as desired. Techniques for attachment include those described in U.S. Pat. No. 4,376,110 and the references cited therein. If covalent, the plate or other solid phase is incubated with a cross-linking agent together with the capture molecules under conditions well known in the literature.

Commonly used cross-linking agents for attaching the capture molecules to the solid-phase substrate include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-((p-azidophenyl)-dithio)propioimidate yield photoactivatable intermediates capable of forming cross-links in the presence of light.

The coated plates are then typically treated with a blocking agent that binds non-specifically with and saturates the unoccupied binding sites to prevent unwanted binding of the free ligand to the excess sites on the wells of the plate. Examples of appropriate blocking agents for this purpose include, e.g., gelatin, bovine serum albumin, egg albumin, casein, and non-fat milk. The blocking treatment typically takes place under conditions of ambient temperatures for about 1-4 hours, typically about 1.5 to 3 hours.

After coating and blocking, the calibrator formulations (known concentrations of MPO isoforms or proforms that act as calibrators) or the test sample to be analyzed, appropriately diluted, are added to the immobilized phase. Commonly, the dilution rate is about 5-15% (v/v).

The amount of capture molecule employed is sufficiently large to give a good signal in comparison with the calibration standards, but is generally not in molar excess compared to the maximum expected level of MPO isoform and/or proform or combination thereof that is of interest in the sample. For sufficient sensitivity, the amount of test sample should be added such that the immobilized capture molecules are in molar excess of the maximum molar concentration of free analyte of interest anticipated in the test sample after appropriate dilution of the sample. This anticipated maximum level depends mainly on any known correlation between the concentration levels of MPO isoforms and/or proforms of interest in the particular test sample being analyzed and the clinical condition of the patient.

Generally, the conditions for incubation of sample and immobilized capture molecule are selected to maximize analytical sensitivity of the assay to minimize dissociation, and to ensure that sufficient analyte of interest that is present in the sample binds with the immobilized capture molecule. It is understood that the selection of optimum reaction conditions generally requires only routine experimentation. The incubation is accomplished at fairly constant temperatures, ranging from about 0° C. to about 40° C., generally at or about room temperature. The time for incubation is generally no greater than about 10 hours. The duration of incubation may be longer if a protease inhibitor is added to prevent proteases in the test sample from degrading the MPO isoforms or proforms of interest.

The detector molecule can be labeled with any detectable marker that does not interfere with the binding of the captured analyte to the detecting agent. Examples of suitable label moieties include moieties that may be detected directly, such as fluorochrome, chemiluminescent, colorimetric, energy transfer agents and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase bacterial luciferase (U.S. Pat. No. 4,737,456), luciferin, 2,3-dihydrophthalazinediones, HRP, alkaline phosphatase, amylase, catalase, lactamase, hexokinase, urease, malate dehydrogenase, P-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, biotin/streptavidin, biotin/Streptavidin-p-galactosidase with 4-methylumbelliferyl-d-galactosidase (MUG), spin labels, bacteriophage labels, stable free radicals, and the like.

Conjugation of the label moiety to the detection molecule, such as for example an antibody, is a standard manipulative procedure in immunoassay techniques. See, for example, O'Sullivan et al., 1981, “Methods for the Preparation of Enzyme-antibody Conjugates for Use in Enzyme Immunoassay,” in Methods in Enzymology, Langone and Van Vunakis, Eds., Vol. 73 (Academic Press, New York, N.Y.), pp. 147-166. Conventional methods are available to bind the label moiety covalently to proteins or polypeptides. For example, coupling agents such as dialdehydes, carbodiimides, dimaleimides, bis-imidates, bis-diazotized benzidine, and the like, may be used to label antibodies with the above-described fluorescent, chemiluminescent, and enzyme labels. See, for example, U.S. Pat. No. 3,940,475 (fluorimetry) and U.S. Pat. No. 3,645,090 (enzymes); Hunter et al., Nature, 144:945 (1962); David et al., Biochemistry, 13:1014-1021 (1974); Pain et al., J. Immunol Methods, 40:219-230 (1981); and Nygren J., Histochem. and Cytochem., 30:407-412 (1982). Fluorescent or chemiluminescent labels can be used to increase amplification and sensitivity to about 5-10 pg/ml.

Not to be considered limiting, the amount of MPO isoform and/or proform analyte bound to the capture molecule can be determined by washing away unbound detection molecule from the immobilized phase, and measuring the amount of detection molecule bound to the analyte using a detection method appropriate to the label. The label moiety can be, for example, an enzyme. In the case of enzyme moieties, the amount of developed signal, for example, color, is a direct measurement of the amount of captured analyte. For example, when HRP is the label moiety, color is detected by quantifying the optical density (O.D.) at 450 nm absorbance. In another embodiment, the quantity of analyte bound to the capture molecule can be determined indirectly. The signal of an unlabeled detection molecule may be amplified for detection with a detection molecule-specific antibody conjugated to a label moiety. For example, the signal of an unlabeled mouse antibody that binds with the analyte may be amplified with a mouse IgG-specific sheep antibody labeled with HRP. The label moiety is detected using a detection method appropriate to the label. For example, HRP may be detected by reacting HRP with a calorimetric substrate and measuring the optical density of the reacted substrate at 450 nm absorbance.

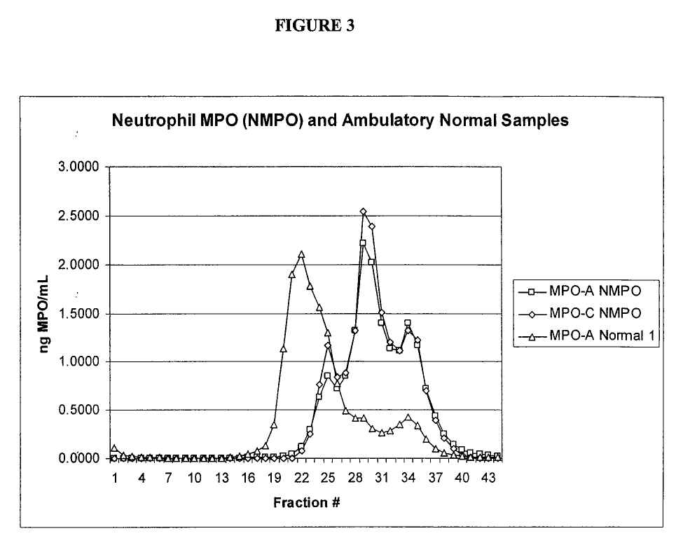

Following differential detection of the at least one MPO isoform(s) and/or proforms within the test sample by differential detection, the presence or lack of the particular isoforms or proforms can be correlated with specific allergic, inflammatory, cardiovascular or autoimmune disease conditions. By “differential detection” is meant detecting the presence/quantity of one or more specific isoforms or proforms relative to total MPO, including all active and inactive forms, or alternatively by detecting the presence/quantity of one or more specific isoforms or proforms without detection of inactive forms, such as an excreted proform of MPOs. In a further embodiment, reactivity of the capture molecules to all native isoforms of MPO is excluded in the detection of MPO isoforms and proforms (see, e.g. fractions 19-25 of FIG. 2 and FIG. 3). In a further embodiment, reactivity of the capture molecules to proforms of MPO is excluded in the detection. By “correlate” or “correlating” is meant comparing, in any way, the performance and/or results of a first analysis with the performance and/or results of a second analysis. For example, as described in greater detail below, MPO expressed by normal and diseased individuals differs in the expression of MPO isoforms and/or proforms. By identifying a particular isoform and/or proform whose expression is elevated in a disease condition, the isoform and/or proform can act as an indicator or as a predictor of a specific disease condition, thereby “correlating” isoform and/or proform expression with disease condition status. In certain embodiments of the disclosure, data obtained from an immunoassay using calibration standards are correlated with data obtained by measurement of a MPO isoform and/or proform, for example in the form of optical density, in order to quantitatively detect a MPO isoform and/or proform in a sample.

The method also provides for the quantitation of individual MPO isoform and/or proform levels in a test sample and correlation of said levels to a disease condition. In one embodiment, the relative levels of the individual MPO isoforms and/or proforms in a test sample are determined and said levels are correlated to the presence of, or propensity for a specific disease condition. For example, an increase in the level of MPO isoform X and a decrease in the level of isoform Y are indicative of the presence of, or future development of disease condition 1, while an increase in the level of isoform Y and an increase in the level of isoform Z are indicative of the presence of, or future development of disease condition 2. In other instances, the relative ratios of, for example, isoform X and isoform Z, are indicative of the presence of, or future development of disease condition 3. It is also recognized that levels and/or ratios of three or more isoforms and/or proforms can be used as indicators of the presence of, or future development of a disease condition.

### Kits

As a matter of convenience, the assay method of this disclosure can be provided in the form of a kit. Such a kit is a packaged combination comprising the basic elements of: (a) at least one capture molecule that specifically binds with at least one MPO isoform and/or proform of interest; (b) at least one detector molecule that either binds with at least one MPO isoform and/or proform, or is a labeled MPO isoform or proform or combination thereof; and (c) instructions on how to perform the assay method using these reagents.

In one embodiment, the kit may further provide calibrators of MPO isoforms or proforms useful in the correlation of the isoforms or proforms with specific allergic, inflammatory, cardiovascular or autoimmune disease conditions. The calibrators are generally known quantities of MPO isoform or proform polypeptides or combinations thereof that can be isolated from cells naturally expressing these isoforms or proforms, or polypeptides produced synthetically or recombinantly using techniques known in the art.

In another embodiment, the kit further comprises a solid support for the capture molecules, which may be provided as a separate element or as an element on which the capture molecules are already immobilized. Hence, the capture molecules in the kit either may be immobilized already on a solid support, or may become immobilized on a support that is included with the kit or provided separately from the kit. The detector molecules may be labeled molecules (e.g. antibodies) detected directly or unlabeled molecules that are detected by labeled antibodies directed against the unlabeled antibodies raised in a different species. Where the label is an enzyme, the kit will ordinarily include substrates and cofactors required by the enzyme, where the label is a fluorophore, a dye precursor that provides the detectable chromophore, and where the label is biotin, an avidin such as avidin, streptavidin, either alone or conjugated to a chromophore.

The kit may further include an instruction sheet, describing how to carry out the assay of the kit.

### EXAMPLES

### Example 1

### Detection of MPO Isoforms and Proforms

Two sandwich assay formats, namely MPO-A and MPO-C, were used in the development of the assays for specific detection of one or more isoforms and/or proforms.

Both MPO-A and MPO-C assays utilized the capture antibody 16E3 (Hytest, Turku, Finland). The capture antibody was coupled with biotin and then secondarily coupled with streptavidin-coated paramagnetic particles. The detection molecules were conjugate antibodies that were coupled to alkaline phosphatase. The conjugate antibody for MPO-A is PV 722 and for MPO-C is PV 723 (both from Diagnostics Development, Uppsula, Sweden).

For detecting the MPO isoforms and/or proforms, the test sample was diluted 1:20 in 0.1 M TRIS, 0.15 M NaCl, 2 mM MgCl₂, 0.1 mM ZnCl₂, pH 7.0 @ 37° C. prior to analysis. The sample was then incubated with the coated paramagnetic particle, conjugate antibody and blocking reagents for 5 minutes at 37° C. The blocking reagent contains sample diluent and 1 mg/mL goat IgG, 1 mg/mL mouse IgG, 0.05 mg/mL Poly Mak IgG (from Roche), 0.02 mg/mL inactivated alkaline phosphatase, 0.1% Proclin 300 (from Sigma Aldrich), 0.2 M MgCl₂. The sample preparation was then subjected to a magnetic field and washings to remove materials not bound to the solid phase. The bound materials were then incubated for 5 minutes at 37° C. with the chemiluminescent substrate Lumi-Phos 530 from Lumigen. Light emission was detected with a photomultiplier tube using the Access® instrument (from Beckman Coulter). The amount of MPO isoform and/or proform or combination thereof in the sample was determined using a stored multipoint calibration curve.

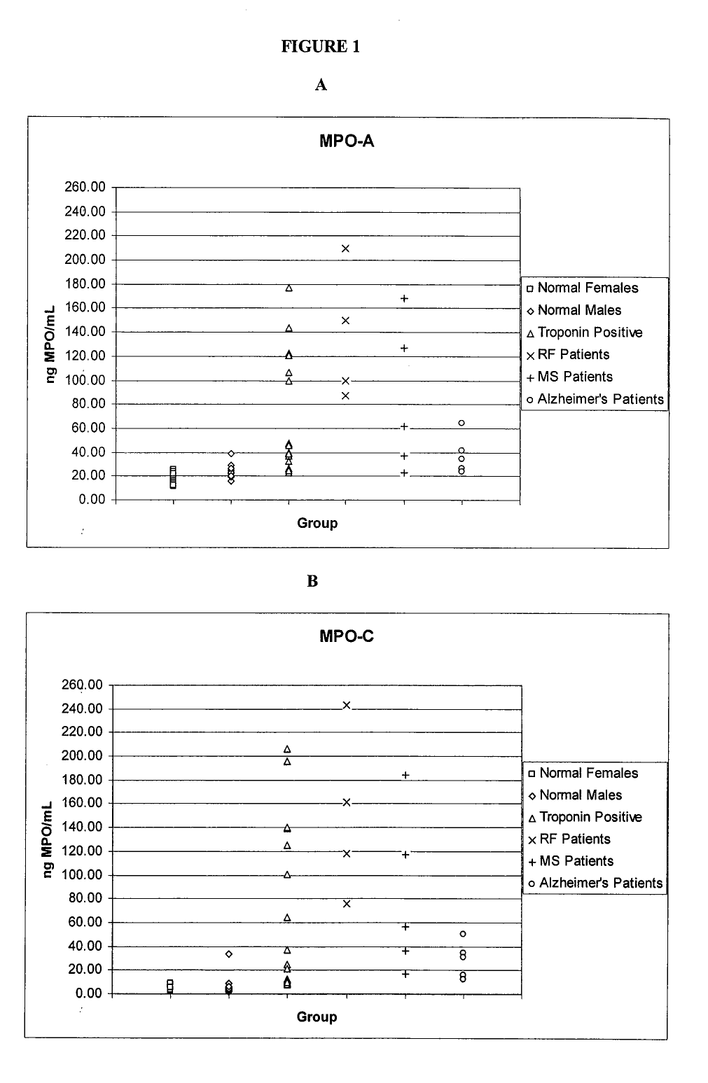

Depending on the antibodies used for the different assays, specific patient samples showed significantly different results (FIG. 1). This difference is especially notable in the ambulatory normal samples. The significantly different results from using specific patient samples in different assay formats are believed due to differential reactivity of the antibodies to the MPO isoforms or proforms. This led to understanding that conventional detection of MPO, similar to that shown in the MPO-A assay, quantified all MPO isoforms and proforms equally, irregardless to whether the isoforms are active or inactive. Thereby resulting in low specificity for diagnosing selected disease states.

This resulted in development of assay methods more specific for a number of MPO isoforms and/or proforms or combinations thereof to improve diagnostic specificity, or to improve sensitivity by reducing the normal cutoff.

Column chromatography techniques have been optimized to fractionate individual patient sample MPO into isoforms and/or proforms which have been measured using sensitive immunoassays. Chromatography was performed as follows:

| | |
|---|---|
| Column: | TOSOH SP 5 PW cation exchange column. 7.5 mm × 7.5 cm |
| Buffer A: | 0.02 M PO₄, pH 7.3 |
| Buffer B: | 0.02 M PO₄, 0.15 M NaCl, 2 M KCl, pH 6.7 |

The initial column buffer was 6% buffer B in buffer A. The flow rate was 0.75 ml/min. The test sample was applied to the equilibrated column followed by 2 column volumes of the initial column buffer. A gradient was collected up to 50% buffer B in buffer A. The gradient required 7 column volumes to complete. Forty-six 0.5 ml fractions were collected during the elution gradient. The collected fractions were assayed using the MPO-A and MPO-C formats on Access® without prior dilution. Non-fractionated samples were diluted 1:20 prior to assaying. The fractions were tested undiluted because the column diluted the sample in the process of binding and eluting the MPO isoforms or proforms. By testing fractions undiluted, the assay has essentially 20-fold more sensitivity.

Both unfractionated and column chromatography-fractionated samples from ambulatory normal and cardiac Troponin I (a known marker of cardiac tissue damage) positive patients have been measured using a sensitive immunoassay technique. Unfractionated sample immunoassay results showed that some antibody pairs significantly differed in how they measured the different sample populations (FIG. 1A-B). One pair of antibodies (MPO-C format; FIG. 1B) measured ambulatory normal samples approximately 4-fold lower than a second pair (MPO-A format; FIG. 1A). While the ambulatory normal patient samples read significantly lower in the MPO-C format, nine out of sixteen Troponin I positive samples read approximately as high as, or higher than, in format MPO-A. Additional inflammatory disease state samples including rheumatoid arthritis (RF), multiple sclerosis (MS), and Alzheimer's (AZ) have been measured with these two formats. As with the troponin positive samples, these samples read as high, or higher, in format C, while the ambulatory normal samples read approximately 4-fold lower. The reduced measurement of normal samples while disease state samples remain elevated improves the diagnostic sensitivity of measuring these disease state samples.

Two of the Troponin I positive samples and two of the ambulatory normal samples were fractionated using column chromatography, and the fractions were quantified using the MPO-A assay format (FIG. 2). These data show that the bulk of MPO in the ambulatory normal samples eluted early while most of the MPO in the Troponin I positive samples eluted late. The earlier eluting fractions (fractions 19-25) appear to represent immature MPO proform that is secreted from cells. Additional inflammatory disease state samples including rheumatoid arthritis (RF), multiple sclerosis (MS), and Alzheimer's (AZ) have been fractionated and measured in this way. All of the disease state samples have approximately the same level of early eluting MPO, but are elevated in the later eluting forms.

A sample with MPO from pooled neutrophils (NMPO) was fractionated and the results graphed along with a normal sample (FIG. 3). These data show that the first eluting peak found in both the ambulatory normal and disease state samples is not present in NMPO. These results suggest that in ambulatory normal samples the primary immunoreactive MPO is a different form than the isoforms and/or proforms purified from neutrophils.

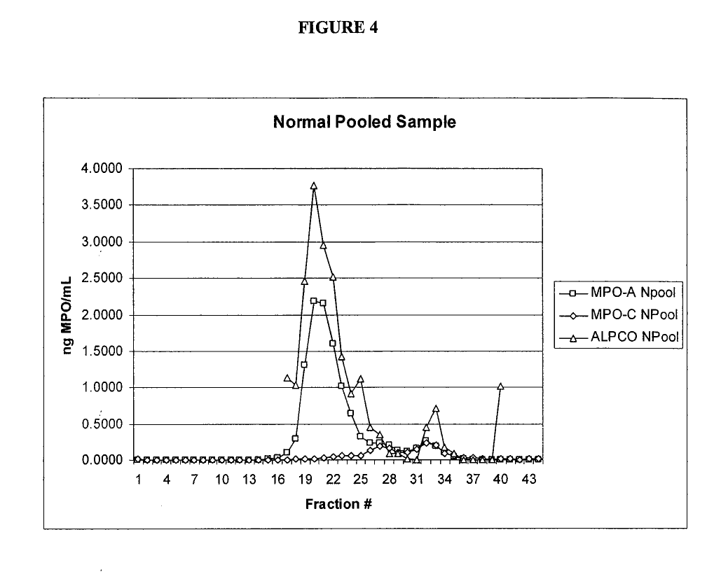

A normal sample pool was fractionated and the fractions were assayed using the MPO-A and MPO-C assay formats, and a competitor method (FIG. 4). The competitor method was that of ALPCO Diagnostics (Windham, N.H.). These results show that the MPO-A format and the competitor kit recognize the earliest eluting peak, while the MPO-C format does not measurably recognize this early peak. These results explain why the MPO-C format reads ambulatory normal samples low, while reading disease state samples equivalent to the MPO-A format. The improved ability of the MPO-C format to discriminate disease samples from ambulatory normal samples is a function of the lack of recognition of the primary, inactive form (see, fractions 19-25 of FIG. 2) found in ambulatory normal samples, while maintaining the ability to measure the forms elevated in disease samples.

In summary, the results of Example 1 show a previously unrecognized ability to differentiate forms (isoforms and/or proforms of MPO and identify subpopulations of MPO to allergic, inflammatory, cardiovascular and/or autoimmune disease states. In addition, this discovery exposes a problem with conventional MPO assays that do not differentiate the isoforms and/or proforms. The results of conventional assays, like MPO-A above, are skewed by detection of an isoform/proform believed to be not related to the above disease states.

### Example 2

### Correlation of MPO Isoforms and/or Proforms with Disease Status

Patient samples are provided in which the patients are suspected of having an autoimmune disease condition. The test samples are subsequently fractionated using ion-exchange chromatography and tested with the MPO-A and MPO-C methods as described above. Several of the test samples demonstrate an MPO isoform and/or proform elution pattern in which the predominant species is an early eluting peak. This pattern indicates that the samples are normal (do not express an MPO isoform and/or proform associated with a disease condition). Several other test samples all contain three predominant late-eluting MPO isoform and/or proform species, with the middle species being the predominant species. The presence of elevated levels of the late-eluting middle species, or of some pattern of elution profile, may be used to identify these samples as being positive for the autoimmune disease condition.

### Example 3

### Correlation of MPO Isoforms and/or Proforms with Therapeutic Efficacy

Patient samples are provided in which the patients are undergoing treatment for an autoimmune disease condition. Associated with this autoimmune disease condition is an elevation in the level of MPO isoform X. Patient samples are obtained throughout the course of treatment (e.g. at the time of diagnosis, during the various stages of treatment, and post-treatment). The samples are processed as described above and subjected to the MPO detection methods described in both the MPO-A and MPO-C format. The test samples are subsequently fractionated using ion-exchange chromatography as described above. Several of the test samples taken during treatment demonstrate an MPO isoform and/or proform elution pattern in which the predominant species is an early eluting peak, while patient-matched samples taken at the time of diagnosis demonstrate late-eluting MPO species. This early eluting pattern indicates that the samples are now normal (do not express an MPO isoform and/or proform associated with a disease condition). Therefore, these results indicate that the patient no longer has the autoimmune disease condition. Several other test samples all contain three predominant late-eluting MPO isoform and/or proform species, with the MPO isoform X as the predominant species. The MPO isoform and/or proform elution pattern from patient-matched samples taken at the time of diagnosis are identical to those taken during the course of treatment. The presence of elevated levels of MPO isoform X is used to identify that these patients currently have the autoimmune disease condition.

While various embodiments of the disclosure have been described above, it should be understood that they have been presented by way of example only, and not limitation. It will be apparent to persons skilled in the relevant art that various changes in form and detail can be made therein without departing from the spirit and scope of the disclosure. Thus, the breadth and scope of the disclosure should not be limited by any of the above-described exemplary embodiments, but should be defined only in accordance with the following claims and their equivalents.

What is claimed is:

1. A method to aid in diagnosis or prognosis of an allergic, inflammatory, cardiovascular or autoimmune disease condition by differentially detecting at least one myeloperoxidase (MPO) isoform or proform or combination thereof in a test sample comprising:
(a) contacting said at least one MPO isoform or proform or combination thereof with at least one capture molecule that specifically binds with said at least one MPO isoform or proform or combination thereof; and
(b) detecting capture of said at least one MPO isoform or proform or combination thereof with at least one detector molecule.

2. The method of claim 1, wherein the captured MPO isoform or proform or combination thereof is detected either by a detector molecule that binds with said at least one MPO isoform or proform or combination thereof, or by competition with a labeled MPO isoform or proform or combination thereof.

3. The method of claim 1, further comprising correlating the detection of said at least one MPO isoform or proform or combination thereof to an allergic, inflammatory, cardiovascular or autoimmune disease.

4. The method of claim 3, wherein said inflammatory disease is inflammatory bowel disease.

5. The method of claim 3, wherein said autoimmune disease is selected from the group consisting of acute disseminated encephalomyelitis, Addison's disease, ankylosing spondylitisis, antiphospholipid antibody syndrome, aplastic anemia, autoimmune hepatitis, coeliac disease, Crohn's disease, diabetes mellitus, Goodpasture's syndrome, Graves' disease, Hashimoto's disease, idiopathic thrombocytopenic purpura, lupus erythematosus, multiple sclerosis, myasthenia gravis, optic neuritis, pemphigus, primary biliary cirrhosis, rheumatoid arthritis, Reiter's syndrome, Sjogren's syndrome, warm autoimmune hemolytic anemia and Wegener's granulomatosis.

6. The method of claim 1, wherein said capture molecule is selected from the group consisting of monoclonal antibody, mono-specific polyclonal antibody, binding fragment of an antibody and antibody mimic.

7. The method of claim 6, wherein said capture molecule is coupled to a solid support comprising a protein coupling surface selected from the group consisting of a microtiter plate, a colloidal metal particle, an iron oxide particle, and a polymeric bead.

8. The method of claim 1, wherein said detector molecule comprises a monoclonal antibody, a mono-specific polyclonal antibody, a binding fragment of an antibody, or an antibody mimic.

9. The method of claim 1, wherein said detector molecule further comprises a detectable label.

10. The method of claim 9, wherein said detectable label comprises a chemiluminescent agent, a calorimetric agent, an energy transfer agent, an enzyme, a substrate of an enzyme reaction, a fluorescent agent or a radioisotope.

11. The method of claim 10, wherein said enzyme is selected from the group consisting of alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase.

12. The method of claim 1, wherein the detector molecule is coupled to a detectable label or to a secondary detector molecule that is coupled to a detectable label.

13. The method of claim 1, wherein said method is an immunoassay.

14. The method of claim 13, wherein said immunoassay is a solid-phase immunoassay.

15. The method of claim 1, wherein said test sample is selected from the group consisting of whole blood, blood plasma, serum, saliva, cerebral spinal fluid, urine, amniotic fluid, interstitial fluid, feces, mucus, cell extracts and tissue extracts.

16. An isolated monoclonal antibody, mono-specific polyclonal antibody, binding fragment of an antibody, or antibody mimic that specifically binds with at least one isoform of MPO, but does not bind all forms of MPO.

17. A kit to aid in diagnosis or prognosis of a disease condition by measuring at least one MPO isoform or proform or combination thereof in a test sample comprising: at least one capture molecule that specifically binds with at least one MPO isoform or proform or combination thereof and at least one detector molecule.

18. The kit of claim 17, wherein the detector molecule is either a molecule that binds with said at least one MPO isoform or proform or combination thereof, or a labeled MPO isoform or proform or combination thereof.

19. The kit of claim 18, wherein the detector molecule is coupled with a detectable label so as to detect capture of said at least one MPO isoform or proform or combination thereof.

20. The kit of claim 17, wherein said disease condition is selected from the group consisting of an allergic, inflammatory, cardiovascular and autoimmune disease.

21. The kit of claim 20, wherein said inflammatory disease is inflammatory bowel disease.

22. The kit of claim 20, wherein said autoimmune disease is selected from the group consisting of acute disseminated encephalomyelitis, Addison's disease, ankylosing spondylitisis, antiphospholipid antibody syndrome, aplastic anemia, antoimmune hepatitis, coeliac disease, Crohn's disease, diabetes mellitus, Goodpasture's syndrome, Graves' disease, Hashimoto's disease, idiopathic thrombocytopenic purpura, lupus erythamatosus, multiple sclerosis, myasthenia gravis, optic neuritis, pemphigus, primary biliary cirrhosis, rheumatoid arthritis, Reiter's syndrom, Sjogren's syndrome, warm autoimmune hemolytic anemia and Wegener's granulomatosis.

23. The kit of claim 17, wherein also is contained a calibration standard for quantization of at least one MPO isoform or proform or combination thereof in a test sample.

24. The kit of claim 17, wherein said capture molecule is selected from the group consisting of monoclonal antibody, mono-specific polyclonal antibody, binding fragment of an antibody, and antibody mimic.

25. The kit of claim 24, wherein said capture molecule is coupled to a solid support comprising a protein coupling surface selected from the group consisting of a microtiter plate, a colloidal metal particle, an iron oxide particle, and a polymeric bead.

26. The kit of claim 17, wherein said detector molecule comprises a monoclonal antibody, a mono-specific polyclonal antibody, a binding fragment of an antibody, or an antibody mimic.

27. The kit of claim 17, wherein said detector molecule further comprises a detectable label.

28. The kit of claim 27, wherein said detectable label comprises a chemiluminescent agent, a colorimetric agent, an energy transfer agent, an enzyme, a substrate of an enzyme reaction, a fluorescent agent or a radioisotope.

29. The kit of claim 28, wherein said enzyme is selected from the group consisting of alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase.

30. The method of claim 1, wherein said method is performed during treatment of a patient having an allergic, inflammatory, cardiovascular or autoimmune disease condition.
